(19) United States
(12) Patent Application Publication
(10) Pub. No.: US 2002/0137154 A1
(43) Pub. Date: Sep. 26, 2002

(54) Methods for improving cell growth and alcohol production during fermentation

(76) Inventors: Lonnie O?apos;Neal Ingram, Gainesville, FL (US); Stuart A. Underwood, Gainesville, FL (US)

Correspondence Address: LAHIVE & COCKFIELD, 28 STATE STREET, BOSTON, MA, 02109, US

(21) Appl. No.: 09/885,294

(22) Filed: Jun. 19, 2001

Related U.S. Application Data
(60) Provisional application No. 60/214,099, filed on Jun. 26, 2000.
(60) Provisional application No. 60/219,844, filed on Jul. 21, 2000.

(51) Int. Cl. C12P 7/06; C12N 1/18

(52) U.S. Cl. 435/161; 435/254.2

(57) ABSTRACT

The invention provides compositions and methods for increasing the production of alcohol, e.g., ethanol, from a saccharide source and increasing the growth of ethanologenic cells by exposing ethanologenic cells to a compound. Preferred alcohologenic cells (cells capable of fermenting a carbon source into an alcohol) are selected from the family Enterobacteriaceae, such as, for example, Escherichia or Klebsiella. Furthermore, the methods and compositions of the invention are suitable for use in simultaneous saccharification and fermentation.

### RELATED INFORMATION

This application claims priority to U.S. provisional application Ser. No. 60/214,099 entitled “Stimulation of Growth and Ethanol Production in Engineered Escherichia Coli Resulting from the Addition of Acetaldehyde” filed Jun. 26, 2000, and U.S. provisional application Ser. No. 60/219,844 entitled “Methods for Improving Cell Growth and Alcohol Production During Fermentation” filed Jul. 21, 2000, both of which are incorporated herein in their entireties by this reference. The contents of all patents, patent applications, and references cited throughout this specification are hereby incorporated by reference in their entireties.

### GOVERNMENT SPONSORED RESEARCH

This work was supported, in part, by U.S. Dept. of Agriculture, National Research Initiative Grant No.:98-35504-6177, and U.S. Dept. of Energy, Basic Energy Sciences, Grant No.:FG02-96ER20222.

### BACKGROUND OF THE INVENTION

Many environmental and societal benefits would result from the replacement of petroleum-based automotive fuels with renewable fuels obtained from plant materials (Lynd et al., (1991) Science 251:1318-1323; Olson et al., (1996) Enzyme Microb. Technol. 18:1-17; Wyman et al., (1995) Amer. Chem. Soc. Symp. 618:272-290). Each year, the United States burns over 120 billion gallons of automotive fuel, roughly equivalent to the total amount of imported petroleum. The development of ethanol as a renewable alternative fuel has the potential to eliminate United States dependence on imported oil, improve the environment, and provide new employment (Sheehan, (1994) ACS Symposium Series No. 566, ACS Press, pp 1-53).

In theory, the solution to the problem of imported oil for automotive fuel appears quite simple. Rather than using petroleum, a finite resource, the ethanol can be produced efficiently by the fermentation of plant material, a renewable resource. Indeed, Brazil has demonstrated the feasibility of producing ethanol and the use of ethanol as a primary automotive fuel for more than 20 years. Similarly, the United States produces over 1.2 billion gallons of fuel ethanol each year. Currently, fuel ethanol is produced from corn starch or cane syrup utilizing either Saccharomyces cerevisiae or Zymomonas mobilis (Z. mobilis). However, both cane sugar and corn starch are relatively expensive starting materials, which have competing uses as food products.

Although some aspects of a biomass conversion process have been demonstrated, ethanol and other chemicals produced from biomass must be cost-competitive with existing petroleum-based products. These costs include nutrients and materials needed for bioconversion, production purification, waste treatment, power, and the manufacturing facility itself.

Therefore, methods that would reduce the cost associated with fermentation, including savings from a reduction in added nutrients and improvements in the rate of production and yield of product, e.g., ethanol, would be beneficial.

### SUMMARY OF THE INVENTION

The present invention provides methods, which overcome the above stated problems of the high cost associated with the production of an alcohol, e.g., ethanol, by fermentation. The invention provides a method for increasing the rate of alcohol production (e.g., ethanol) and the growth of alcohologenic cells (e.g., ethanologenic cells) by contacting or exposing such cells (e.g., by culturing) with a nutrient compound (e.g., a compound of formula I described below) which improves the productivity of the culture (e.g., fermentation rate) and/or growth of the culture (e.g., ability of the cells to grow to a higher cell density or having a reduced cell replication time).

More particularly, in a first aspect, the invention provides a method for increasing production of alcohol from a saccharide source by an alcohologenic cell by, contacting a saccharide source with an alcohologenic cell, and exposing the cell to at least one compound of formula I,

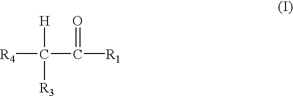

where R₁ is H, OH or COOR₂; R₂ is H or alkyl; R₃ is H, NH₂, alkyl or alkenyl; R₄ is H, alkyl, alkenyl, or a side chain of a naturally occurring amino acid; and salts thereof; where the exposing results in the increased production of alcohol by the alcohologenic cell as compared to a control.

In a second aspect, the invention provides a method for increasing growth of a cell by, contacting a cell with a saccharide source, and exposing the cell to at least one compound of formula I,

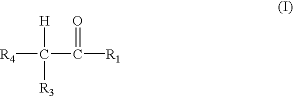

where R₁ is H, OH or COOR₂; R₂ is H or alkyl; R₃ is H, NH₂, alkyl or alkenyl; R₄ is H, alkyl, alkenyl, or a side chain of a naturally occurring amino acid; and salts thereof; where the exposing results in the increased growth of the cell as compared to a control.

In one embodiment of the first two aspects, the compound of formula I is a lower aliphatic aldehyde, lower aliphatic α-keto carboxylic acids, lower aliphatic dicarboxylic acid, amino acid, or salt of any of the foregoing acids.

In one embodiment of the first aspect, the alcohol is ethanol and the alcohologenic cell is an ethanologenic cell. In a related embodiment, the increased production of ethanol is indicated by an increase in volumetric productivity, preferably where the volumetric productivity is between about 0.3 g/L and about 0.5 g/L.

In one embodiment of the above two aspects, the cell is selected from the family Enterobacteriaceae, more preferably, from the genus Escherichia or Klebsiella. In a related embodiment, the cell is E. coli KO4 (ATCC 55123), E. coli KO 11 (ATCC 55124), E. coli KO12 (ATCC 55125), K. oxytoca M5A1, or K. oxytoca P2 (ATCC 55307), LY01 (ATCC______ ). In another related embodiment, the cell is a recombinant cell.

In another embodiment of the above aspects, the compound of formula I is acetaldehyde, pyruvate, succinate, isocitrate, glutamate, α-ketoglutarate, a yeast extract, or casamino acids, and preferably, is acetaldehyde, pyruvate, or glutamate, α-ketoglutarate or a combination thereof.

In a related embodiment, the cell is exposed to glutamate and acetaldehyde, pyruvate and acetaldehyde, fumarate and malate, or α-ketoglutarate and succinate.

In one embodiment, the cell is in an aqueous solution.

In even another embodiment, the saccharide source is cellooligosaccharide, lignocellulose, hemicellulose, cellulose, pectin, xylose, glucose, corn steep liquor (CSL), or any combination thereof.

In another embodiment, the cell is exposed to the compound of formula I for a period of time between about 1 and about 96 hours.

In another embodiment, the method is performed at a pH between about 6 and about 8, and preferably at a pH of about 6.5.

In another embodiment, the method is performed at a temperature between about 20° and about 40° C., and preferably at a temperature of about 35° C.

In another embodiment, the compound is present at a concentration between about 0.1 and about 4.0 g/L.

In another embodiment, the method of the above aspects further includes exposing the cell to the compound more than once. In a related embodiment, the exposing of the cell to the compound is performed at time intervals between about 1 hour and about 24 hours.

In another embodiment, the method of the above aspects further includes exposing the cell to two or more different compounds of formula I.

In another embodiment, the method of the above aspects further includes agitating the cell, the saccharide source, and the compound between about 50 rpm and about 200 rpm.

In one embodiment of the second aspect, the increased growth is indicated by increased cell density or decreased cell replication time. In a related embodiment, the increased cell density is indicated by an optical density of between about 2 and about 3 at 550 nm after 24 hours.

In another embodiment, the method of the above aspects is performed in a fermentor vessel, where, preferably, the cell and the saccharide source are provided in an aqueous solution. In a related embodiment, the aqueous solution includes a fermentation medium, preferably Luria broth or CSL broth.

In yet another embodiment, the method of the above aspects is suitable for simultaneous saccharification and fermentation.

In a third aspect, the present invention provides a growth medium suitable for use in an improved fermentation process including a saccharide source, a basal nutrient medium, and at least one compound of formula I,

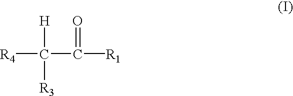

where R₁ is H, OH or COOR₂; R₂ is H or alkyl; R₃ is H, NH₂, alkyl or alkenyl; R₄ is H, alkyl, alkenyl; or a side chain of a naturally occurring amino acid; and salts thereof.

In one embodiment, the saccharide source is cellooligosaccharide, lignocellulose, hemicellulose, cellulose, pectin, xylose, glucose, or any combination thereof.

In another embodiment, the basal nutrient medium is Luria broth or CSL broth.

In even another embodiment, the medium is suitable for use in simultaneous saccharification and fermentation.

In still another embodiment, the compound of formula I is acetaldehyde, pyruvate, succinate, citrate, isocitrate, glutamate, α-ketoglutarate, malate, fumarate, a yeast extract, or a casamino acid. In a related embodiment, the compound of formula I is preferably acetaldehyde, pyruvate, succinate, citrate, isocitrate, glutamate, α-ketoglutarate, or malate.

In even another embodiment, the growth medium is packaged with instructions for use.

In a fourth aspect, the invention provides a fermentation reaction mixture suitable for producing ethanol containing, a growth medium having a saccharide source, an ethanologenic cell, and an exogenous source of at least one compound of formula I,

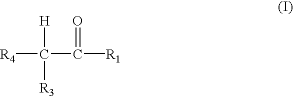

where R₁ is H, OH or COOR₂; R₂ is H or alkyl; R₃ is H, NH₂, alkyl or alkenyl; R₄ is H, alkyl, alkenyl; or a side chain of a naturally occurring amino acid; and salts thereof.

In one embodiment, the fermentation reaction mixture includes a saccharide source selected from the group consisting of cellooligosaccharide, lignocellulose, hemicellulose, cellulose, pectin, xylose, glucose, or any combination thereof.

In another embodiment, the ethanologenic cell of the fermentation reaction mixture is from the family Enterobacteriaceae.

In another embodiment, the fermentation reaction mixture is suitable for use in simultaneous saccharification and fermentation.

In still another embodiment, the compound of formula I is acetaldehyde, pyruvate, succinate, isocitrate, glutamate, α-ketoglutarate, fumarate, a yeast extract, or a casamino acid, and preferably, acetaldehyde, pyruvate, succinate, isocitrate, glutamate, or α-ketoglutarate.

Advantages of the above compositions and methods include the ability to reduce the overall cost of biomass conversion to a useable fuel. For example, increases in alcohol yield or alcohol titer provide the benefits of reducing the amount of biomass which must be treated accompanied by corresponding reductions in the costs of feedstocks, chemicals, equipment, and energy throughout the process. Improvements in yield and titer also reduces the amount of waste generated and the costs associated with waste disposal.

Other features and advantages of the invention will be apparent from the following detailed description and claims.

### BRIEF DESCRIPTION OF THE DRAWINGS

FIG. 1 shows ethanol production and cell growth by ethanologenic bacteria when cultured in broth containing 1% corn steep liquor (CSL), xylose, salts and different amounts of an additional nutrient compound (i.e., acetaldehyde) as compared to a control. Panel A shows ethanol production in g/L over time (96 hours), and panel B shows changes in cell growth (measured as cell mass at OD₅₅₀ₙₘ) over time (96 hours). Ethanol production and cell mass are determined at several time points from the start of fermentation to the end of the 96 hour time period. The additional nutrient compound acetaldehyde was added to the fermentation medium at five different concentrations and at five different time points as indicated.

FIG. 2 shows ethanol production and cell growth by ethanologenic bacteria when cultured in broth containing 1% corn steep liquor (CSL), glucose, salts, and different amounts of an additional nutrient compound (i.e., acetaldehyde) as compared to a control. Panel A shows ethanol production in g/L over time (96 hours), and panel B shows changes in cell growth (measured as cell mass at OD₅₅₀ₙₘ) over time (96 hours). Ethanol production and cell mass are determined at several time points from the start of fermentation to the end of the 72 hour time period. The additional nutrient compound acetaldehyde was added to the fermentation medium at five different concentrations and at five different time points as indicated.

FIG. 3 shows ethanol production and cell growth by ethanologenic bacteria when cultured in Luria broth containing xylose and different amounts of an additional nutrient compound (i.e., acetaldehyde) as compared to a control. Panel A shows ethanol production in g/L over time (72 hours), and panel B shows changes in cell growth (measured as cell mass at OD₅₅₀ₙₘ) over time (72 hours). Ethanol production and cell mass are determined at several time points from the start of fermentation to the end of the 72 hour time period. The additional nutrient compound acetaldehyde was added to the fermentation medium at five different concentrations and at five different time points as indicated.

FIG. 4 is a schematic representation of the sugar to ethanol pathway indicating acetaldehyde as an intermediate metabolite in the pyruvate to ethanol pathway.

FIG. 5 is a schematic representation of the overall fermentation pathway for hexoses and pentoses.

### DETAILED DESCRIPTION OF THE INVENTION

Before further description of the invention, certain terms employed in the specification, examples and appended claims are, for convenience, collected here.

I. Definitions

As used herein, the term “medium” or “media”, refers to an aqueous or solid source of nutrients capable of supporting the growth of a cell, preferably, for example, an alcohologenic cell capable of fermenting a carbon source, such as a sugar into an alcohol. Examples of media include, e.g, Luria broth (LB), NZCYM medium, NZYM medium, NZM medium, SOB medium, SOC medium, ZXYT medium, M9 minimal medium, Terrific broth (TB) (see also, Sambrook et al., Molecular Cloning: A Laboratory Manual, CSHL Press (1989); Ausubel et al., Current Protocols in Molecular Biology, Wiley Interscience).

The term “Luria broth” or “LB” includes media typically comprising a yeast extract (e.g., crude, self digested solubles from yeast bodies containing, e.g., amino acids, peptides, vitamins, lipids, nucleosides, salts, etc.), casamino acids (i.e., an enzymatic digestion of casein protein comprising amino acids and peptides), and salts (e.g., sodium chloride).

The term “CSL medium” includes a medium typically comprising corn steep liquor, a fermentable sugar, and a mixture of salts essential for growth.

The term “cell”, refers to the smallest structure capable of independently carrying out life sustaining processes, including metabolic processes, e.g., growth, and reproduction. The term “cell,” as used herein, includes a bacterial, yeast, fungal, plant, or animal cell.

The term “nutrient compound”, includes any molecule or compound, added to the medium of a cell during the culture of the cell for the purpose of improving product yield or cell growth as compared to a control. The term includes any compound of the formula I:

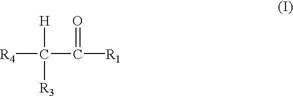

wherein;

R₁ is H, OH or COOR₂;

R₂ is H or alkyl;

R₃ is H, NH₂, alkyl or alkenyl;

R₄ is H, alkyl, alkenyl, or a side chain of a naturally occurring amino acid, and salts thereof. Preferred nutrient compounds of formula I include but are not limited to, lower aliphatic aldehydes, lower aliphatic α-keto carboxylic acids, lower aliphatic dicarboxylic acids, amino acids, and salts of any of these acids. Preferably, carboxylic acid compounds of formula I are used as salts, e.g., mono- or bi-potassium and/or sodium salts, hydrated or unhydrated. Particularly preferred compounds of formula I are those listed in Tables 1-7, including but not limited to, acetaldehyde, pyruvate, glutamate, aspartate, isocitrate, oxaloacetate, alanine, succinate, fumarate, malate, α-ketoglutarate, yeast extract, and amino acids, e.g, casamino acids, separately or in any combination.

The term “alkyl” is art-recognized and includes the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), more preferably 20 or fewer, and still more preferably four or fewer. Likewise, preferred cycloalkyls have from four to ten carbon atoms in their ring structure, and more preferably have 5, 6, or 7 carbons in the ring structure.

Unless the number of carbons is otherwise specified, the term “lower” as in “lower alkyl” and/or “lower aliphatic” is intended to denote a saturated or unsaturated aliphatic hydrocarbon (e.g., alkyl or alkenyl as defined herein) having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl, and so forth. Likewise, “lower alkenyl” and “lower alkynyl” have similar chain lengths. Preferred alkyl groups include lower alkyls. Examples of alkylene groups are methylene, ethylene, propylene, and so forth.

Moreover, the term alkyl as herein is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, carbonyl (including aldehydes, ketones, carboxylates, and esters), alkoxyl, ether, phosphoryl, cyano, amino, acylarnino, amido, amidino, imino, sulfhydryl, alkylthio, arylthio, thiolcarbonyl (including thiolformates, thiolcarboxylic acids, and thiolesters), sulfonyl, nitro, heterocyclyl, aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, acylaminos, iminos, amidos, phosphoryls (including phosphonates and phosphinates), sulfonyls (including sulfates, sulfonatos, sulfamoyls, and sulfonamidos), and silyl groups, as well as ethers, alkylthios, arylthios, carbonyls (including ketones, aldehydes, carboxylates, and esters), —CF₃, —CN, and the like. Exemplary substituted alkyls are described below. Cycloalkyls can be further substituted with alkyls, alkenyls, alkoxys, alkylthios, arylthios, aminoalkyls, carbonyl-substituted alkyls, —CF₃, cyano (—CN), and the like.

The term “aralkyl”, as used herein, refers to an alkyl group substituted with an aryl group (e.g., an aromatic or heteroaromatic group).

The terms “alkenyl” and “alkynyl” are art-recognized and include unsaturated aliphatic groups analogous in length and possible substitution to the alkyls described above, but that contain at least one double or triple bond, respectively.

The term “alkoxyl” is art-recognized and includes any group represented by the formula —O-alkyl. Representative alkoxyl groups include methoxy, ethoxy, propoxy, tert-butoxy, and the like. Unless otherwise specified, an “alkoxy” group can be replaced with a group represented by —O-alkenyl, —O-alkynyl, —O-aryl (i.e., an aryloxy group), or —O-heterocyclyl. An “ether” is two substituted or unsubstituted hydrocarbons covalently linked by oxygen. Accordingly, the substituent of, e.g., an alkyl that renders that alkyl an ether is, or resembles, an alkoxyl, such as can be represented by one of —O-alkyl, —O-alkenyl, —O-alkynyl, —O-aryl, or —O-heterocyclyl. The term “lower alkoxy” includes a lower alkyl group attached to the remainder of the molecule by oxygen.

Examples of alkoxy groups include methoxy, ethoxy, isopropoxy, tert-butoxy and so forth. The term “phenyl alkoxy” refer to an alkoxy group, which is substituted by a phenyl ring. Examples of phenyl alkoxy groups are benzyloxy, 2-phenylethoxy, 4-phenylbutoxy, and so forth. The term “alkanoyloxy group” refers to the residue of an alkylcarboxylic acid formed by removal of the hydrogen from the hydroxyl portion of the carboxyl group. Examples of alkanoyloxy groups include formyloxy, acetoxy, butyryloxy, hexanolyoxy, and so forth. The term “substituted” as applied to “phenyl” refers to phenyl which is substituted with one or more of the following groups: alkyl, halogen (i.e., fluorine, chlorine, bromine or iodine), nitro, cyano, trifluoromethly, and so forth. The “alkanol” or a “hydroxyalkyl” refer to a compound derived by protonation of the oxygen atom of an alkoxy group. Examples of alkanols include methanol, ethanol, 2-propanol, 2-methyl-2-propanol, and the like.

The term “halogen” designates —F, —Cl, —Br or —I; the term “sulfhydryl” or “thiol” means —SH; the term “hydroxyl” means —OH.

The term “aryl” is art-recognized and includes 5- and 6-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles”, “heteroaryls”, or “heteroaromatics”. The aromatic ring can be substituted at one or more ring positions with such substituents as described above, as for example, halogen, alkyl, aralkyl, alkenyl, alkynyl, cycloalkyl, hydroxyl, amino, acylamino, azido, nitro, sulfhydryl, imino, amido, amidino, phosphonate, phosphinate, carbonyl, carboxyl, silyl, ether, alkylthio, arylthio, sulfonyl, sulfonamido, sulfamoyl, ketone, aldehyde, ester, a heterocyclyl, an aromatic or heteroaromatic moiety, —CF₃, —CN, or the like. Aryl groups can also be fused or bridged with alicyclic or heterocyclic rings, which are not aromatic so as to form a polycycle (e.g., tetralin).

The term “amino acid” includes its art recognized meaning and broadly encompasses compounds of formula II:

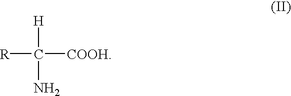

Preferred amino acids include the naturally occurring amino acids, as well as synthetic derivatives, and amino acids derived from proteins, e.g., proteins such as casein, i.e., casamino acids, or enzymatic or chemical digests of, e.g., yeast, an animal product, e.g., a meat digest, or a plant product, e.g., soy protein, cottonseed protein, or a corn steep liquor (see, e.g., Traders' Guide to Fermentation Media, Traders Protein, Memphis, Tenn. (1988), Biotechnology: A Textbook of Industrial Microbiology, Sinauer Associates, Sunderland, Mass. (1989), and Product Data Sheet for Corn Steep Liquor, Grain Processing Corp., IO). The term “naturally occurring amino acid” includes any of the 20 amino acid residues which commonly comprise most polypeptides in living systems, rarer amino acids found in fibrous proteins (e.g., 4-hydorxyproline, 5-hydroxylysine, ε-N-methyllysine, 3-methylhistidine, desmosine, isodesmosine), and naturally occurring amino acids not found in proteins (e.g., β-alanine, γ-aminobutryic acid, homocysteine, homoserine, citrulline, ornithine, canavanine, djenkolic acid, and β-cyanoalanine).

The term “side chain of a naturally occurring amino acid” is intended to include the side chain of any of the naturally occurring amino acids, as represented by R in formula II. One skilled in the art will understand that the structure of formula II is intended to encompass amino acids such as proline where the side chain is a cyclic or heterocyclic structure (e.g., in proline R group and the amino group form a five-membered heterocyclic ring. Similarly, the compound of formula I above is intended to encompass amino acids such as proline wherein in formula I, e.g, R3 and R4 form a heterocyclic ring.

The term “increasing production of alcohol” refers to any increase in the yield of alcohol, e.g., ethanol, the volumetric productivity of a fermentation reaction, or the rate of production of alcohol, e.g., ethanol, from a fermentation reaction over a certain period of time or at the completion of the fermentation reaction, as compared to a control.

The term “volumetric productivity” includes the increased productivity of a cell culture where the productivity of the cells is typically measured as an increase in the amount of a cell derived product in a given cell culture volume, preferably, e.g., an increase in the amount of alcohol produced in grams per liter of culture (i.e., g/L).

The term “increasing growth of a cell” includes increased cell density or cell mass, and/or decreased cell replication time as compared to a control. Cell mass and cell density may be determined by the optical density (OD) of the cells in suspension at any given time point.

The term “exposing” includes contacting the cell with a nutrient compound, e.g., acetylaldehyde from any source. The cell may or may not be in aqueous solution.

The term “fermentation reaction” refers to any mixture of medium and cells capable of fermenting a saccharide source.

The term “fermentor vessel” refers to any container capable of supporting a fermentation reaction. A fermentor vessel may be capable of containing a volume of between 0.10 to 100 L, or more (e.g., 1,000,000 L). A fermentor vessel may also have a means of controlling temperature and pH and may provide a source of agitation (e.g., via an impeller and/or sparging) for the contents of the vessel. In addition, a fermentor vessel may also provide a source of gas flow (oxygen, nitrogen, and/or carbon dioxide). Typically a fermentor vessel allows for all or some of the foregoing culture characteristics or parameters to be advantageously monitored and/or controlled.

The term “exogenous source” is intended to include any source of a nutrient compound that is added to the fermentation reaction. Examples of exogenous sources of nutrient compounds are described in the Examples.

The term “basal nutrient medium” is any medium, which contains all of the elements essential for maintaining the fundamental vital activities of an organism. For example, a basal nutrient medium includes Luria broth (LB).

The term “recombinant cell” is intended to include a genetically modified cell. The cell can be a microorganism or a higher eukaryotic cell. The term is intended to include progeny of the cell originally modified. In preferred embodiments, the cell is a alcohologenic bacterial cell, e.g., a Gram-negative bacterial cell, and this term is intended to include all facultatively anaerobic Gram-negative cells of the family Enterobacteriaceae such as Escherichia, Shigella, Citrobacter, Salmonella, Klebsiella, Enterobacter, Erwinia, Kluyvera, Serratia, Cedecea, Morganella, Hafnia, Edwardsiella, Providencia, Proteus, and Yersinia. Particularly preferred recombinant hosts are Escherichia coli or Klebsiella oxytoca cells having alcohologenic activities. More preferred host cells have polysaccharase and alcohologenic activities and can ferment a complex sugar. Examples of such cells are provided in U.S. Pat. Nos. 5,821,093; 5,482,846; 5,424,202; 5,028,539; 5,000,000; 5,487,989, 5,554,520; 5,162,516; and U.S. Ser. No. 60/136,376.

The term “polysaccharase” includes a polypeptide capable of catalyzing the degradation or depolymerization of any linked sugar moiety, e.g., disaccharides, trisaccharides, oligosaccharides, including, complex carbohydrates, i.e., complex sugars, e.g., lignocellulose, which comprises cellulose, hemicellulose, and pectin. The terms are intended to include cellulases such as glucanases, including both endoglucanases and exoglucanases, and β-glucosidase.

The term “complex sugar” includes any carbohydrate source comprising more than one sugar molecule. These carbohydrates may be derived from any unprocessed plant material or any processed plant material. Examples are wood, paper, pulp, plant derived fiber, or synthetic fiber comprising more than one linked carbohydrate moiety, i.e., one sugar residue. One particular complex sugar is lignocellulose, which represents approximately 90% of the dry weight of most plant material and contains carbohydrates, e.g., cellulose, hemicellulose, pectin, and aromatic polymers, e.g., lignin. Cellulose makes up 30%-50% of the dry weight of lignocellulose and is a homopolymer of cellobiose (a dimer of glucose).

The term “saccharide source” includes any sugar including, for example, monosaccharides, disaccharides, oligosaccharides, complex sugars, or any combination thereof. Exemplary saccharide sources include, e.g, glucose and xylose. Any one or a combination of the above carbohydrates are potential sources of sugars for depolymerization (if needed) and subsequent bioconversion to an alcohol, e.g., ethanol, by fermentation according to the present invention.

The term “simultaneous saccharification and fermentation” or “SSF” is intended to include the use of one or more cells, e.g., recombinant cells, for the contemporaneous degradation or depolymerization of a complex sugar and bioconversion of that sugar into an alcohol, e.g., ethanol, by fermentation.

The term “ethanologenic” is intended to include the ability of a microorganism to produce ethanol from a carbohydrate as a primary fermentation product. The term is intended to include naturally occurring ethanologenic organisms, organisms with naturally occurring or induced mutations, and organisms which have been genetically modified.

The term “Gram-negative bacteria” is intended to include the art recognized definition of this term. Typically, Gram-negative bacteria include, for example, the family Enterobacteriaceae which comprises, among others, the species Escherichia and Klebsiella.

The term “alcohologenic” includes the ability of a cell, preferably of a microorganism, to produce an alcohol, e.g., a carbon-based molecule with a hydroxyl moiety, e.g., ethanol, from a carbohydrate as a primary fermentation product. The term is intended to include naturally occurring alcohologenic organisms, organisms with naturally occurring or induced mutations, and organisms which have been genetically modified.

The term “alcohol” refers to any carbon based molecule having a hydroxyl group such as, e.g., ethanol, but also including, e.g., methanol, propanol, butanol, etc.

The term “control” includes its art recognized meaning and, e.g., typically refers to a sample or culture exposed to the same conditions as the test culture but for one parameter such as, e.g., an additional nutrient compound in the medium; i.e., the control sample would not contain the additional nutrient compound, preferably, e.g., a compound represented by formula I, supra.

The term “carbon-based energy source”, “sugar”, or “saccharide source” are used interchangeably and include any sugar that can be metabolized by a cell.

II. Increased Ethanol Production and Cell Growth

The present invention relates, in part, to a method for increasing the rate of alcohol, i.e., ethanol, production and final ethanol titer from a saccharide source by the addition of one or more compounds to fermenting cultures of alcohologenic cells (e.g., ethanologenic cells), as compared to a control with no additional compound. A compound is any of the compounds listed in Tables 1-7, in the Examples. For example, in a preferred embodiment, acetaldehyde and pyruvate are compounds of the invention. Other examples of compounds include, but are not limited to glutamate, aspartate, isocitrate, oxaloacetate, alanine, succinate, fumarate, malate, a-ketoglutarate, yeast extract (an amino source as well as vitamins, minerals, lipids, etc.), and casamino acids (amino acids derived from casein). Compounds of the methods of the invention may be added separately or in any combination.

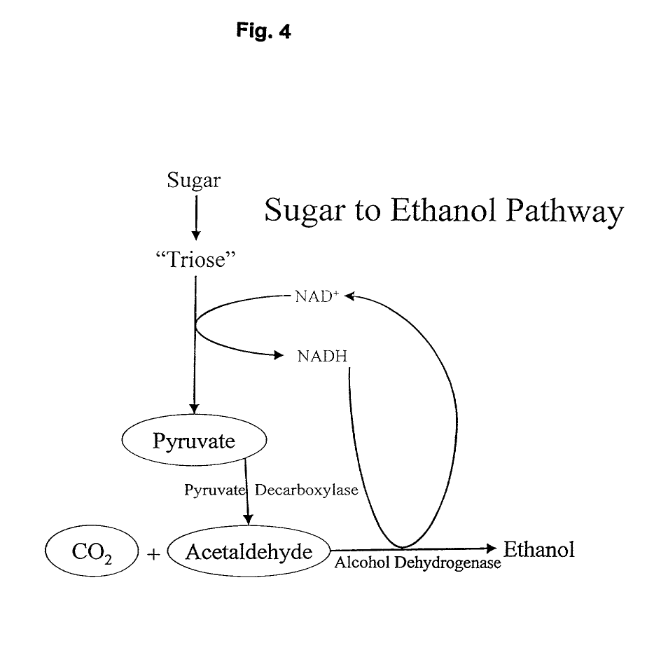

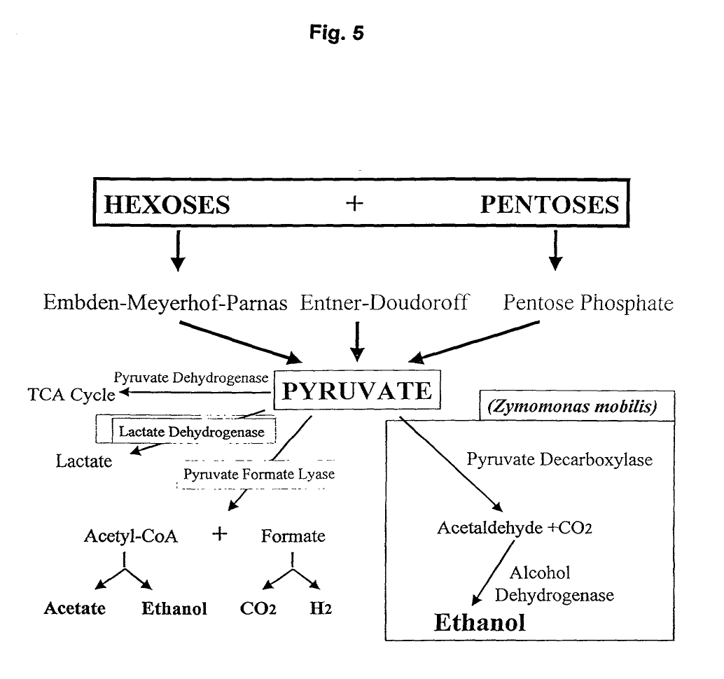

Fermentation products such as ethanol are essentially waste products of sugar metabolism, essential for electron balance and the regeneration of AND+. Acetaldehyde is a product of ethanol-producing microorganisms and an intermediate metabolite in the pyruvate to ethanol pathway (see FIG. 4, and FIG. 5 for a more general schematic). It is produced by the non-oxidative decarboxylation of pyruvate by pyruvate decarboxylase, and subsequently reduced to ethanol during the oxidation of NADH by alcohol dehydrogenase.

Moreover, the present invention relates, in part, to a method of increasing the rate of growth and the final cell concentration achieved in fermenting cultures of cells by the addition of one or more compound listed in Tables 1-7 to the cell culture. Accordingly, an increase in cell growth leads to a higher rate of ethanol production per unit volume during fermentation. Increased growth of the cells may be determined by increased cell density and/or decreased cell replication time. The increase in cell density over time can be used to measure the growth of the cells in culture. Cell mass can be determined by the optical density (OD) of the cells at any given time point. The maximum cell density is the time at which the cell culture has reached the maximum OD. In one embodiment, increased cell density is determined when the cell density is between an optical density of 2 and 3 at 550 nm.

Gas chromatography is advantageously used to measure the increase of ethanol production after addition of a compound as compared to a control. In one embodiment, the production of ethanol is an increase in volumetric productivity. In a preferred embodiment, volumetric productivity is between 0.3 and 0.5 g/L.

In another embodiment of the invention, the method of the invention is performed in a fermentor vessel, allowing for larger volumes of ethanol production from a reduced number of fermentation vessels, and a further reduction of cost in the bioconversion process. A fermentor vessel, as used herein, is any vessel capable of supporting a fermentation reaction.

In one embodiment, the cell used in the methods of the present invention is selected from the family Enterobacteriaceae. For example, the cell may be an Escherichia or a Klebsiella cell. Exemplary E. coli strains that are ethanologenic include, for example, KO4 (ATCC 55123), KO11 (ATCC 55124), and KO12 (ATCC 55125) strains, as well as the LY01 (ATCC______) strain, an ethanol-tolerant mutant of the E. coli strain KO11. Ideally, these strains may be derived from the E. coli strain ATCC 11303, which is hardy to environmental stresses and can be engineered to be ethanologenic and secrete a polysaccharase/s. In addition, recent PCR investigations have confirmed that the ATCC 11303 strain lacks all genes known to be associated with the pathogenicity of E. coli (Kuhnert et al., (1997) AppL. Environ. Microbiol. 63:703-709).

A preferred ethanologenic bacterium is the E. coli KO11 strain which is capable of fermenting hemicellulose hydrolysates from many different lignocellulosic materials and other substrates (Asghari et al., (1996) J. Ind. Microbiol. 16:42-47; Barbosa et al., (1992) Current Microbiol. 28:279-282; Beall et al., (1991) Biotechnol. Bioeng. 38:296-303; Beall et al., (1992) Biotechnol. Lett. 14:857-862; Hahn-Hagerdal et al., (1994) Appl. Microbiol. Biotechnol. 41:62-72; Moniruzzamam et al., (1996) Biotechnol. Lett. 18:955-990; Moniruzzaman et al., (1998) Biotechnol. Lett. 20:943-947; Grohmann et al., (1994) Biotechnol. Lett. 16:281-286; Guimaraes et al., (1992) Biotechnol. Bioeng. 40:41-45; Guimaraes et al., (1992) Biotechnol. Lett. 14:415-420; Moniruzzaman et al., (1997) J. Bacteriol. 179:1880-1886). This strain is able to rapidly ferment a hemicellulose hydrolysate from rice hulls (which contained 58.5 g/L of pentose sugars and 37 g/L of hexose sugars) into ethanol (Moniruzzaman et al., (1998) Biotechnol. Lett. 20:943-947). It was noted that this strain was capable of fermenting a hemicellulose hydrolysate to completion within 48 to 72 hours, and under ideal conditions, within 24 hours.

Another preferred cell used in the methods of the present invention is the bacterium Klebsiella. In particular, Klebsiella oxytoca is preferred because, like E. coli, this enteric bacterium has the native ability to metabolize monomeric sugars, which are the constituents of more complex sugars. Moreover, K. oxytoca has the added advantage of being able to transport and metabolize cellobiose and cellotriose, the soluble intermediates from the enzymatic hydrolysis of cellulose (Lei et al., (1996) Appl. Environ. Microbiol. 63:355-363; Moniruzzaman et al, (1997) Appl. Environ. Microbiol. 63:4633-4637; Wood et al., (1992) Appl. Environ. Microbiol. 58:2103-2110).

In one embodiment, the cell used in the methods of the present invention is a recombinant cell. Accordingly, the methods of the invention provide for use of genetically engineered ethanologenic derivatives of K. oxytoca, e.g., strain M5A1 having the Z. mobilispdc and adhB genes encoded within the PET operon (as described in U.S. Pat. No. 5,821,093; Wood et al., (1992) Appl. Environ. Microbiol. 58:2103-2110). The resulting organism, K. oxytoca P2 (ATCC 55307), produces ethanol efficiently from monomer sugars and from a variety of saccharides including raffinose, stachyose, sucrose, cellobiose, cellotriose, xylobiose, xylotriose, maltose, etc. (Burchhardt et al., (1992) Appl. Environ. Microbiol. 58:1128-1133; Moniruzzaman et aL, (1997) Appl. Environ. Microbiol. 63:4633-4637; Moniruzzaman et al., (1997) J. Bacteriol 179:1880-1886; Wood et al., (1992) Appl. Environ. Microbiol. 58:2103-2110).

In one embodiment, the methods of the present invention are suitable for simultaneous saccharification and fermentation (SSF). SSF is a process in which one or more recombinant hosts are used for the contemporaneous degradation or depolymerization of a complex sugar and bioconversion of that sugar residue into ethanol by fermentation. For example, the strain K. oxytoca P2 is suitable for use in the bioconversion of a complex saccharide in an SSF process as it contains polysaccharase genes in addition to ethanologenic activity (Doran et al., (1993) Biotechnol. Progress. 9:533-538; Doran et al., (1994) Biotechnol. Bioeng. 44:240-247; Wood et al, (1992) Appl. Environ. Microbiol 58:2103-2110). In particular, the use of this ethanologenic P2 strain eliminates the need to add supplemental cellobiase, one of the least stable components of commercial fingal cellulases (Grohmann, (1994) Biotechnol. Lett. 16:281-286). The addition of a nutrient compound to a SSF reaction, according to the methods of the invention, increases the production of ethanol during the simultaneous saccharification and fermentation process.

In another embodiment, the recombinant cell contains a polynucleotide segment that encodes a polysaccharase that is a glucanase, endoglucanase, exoglucanase, cellobiohydrolase, α-glucosidase, endo-1,4-α-xylanase, β-xylosidase, β-glueuronidase, α-L-arabinofuranosidase, acetylesterase, acetylxylanesterase, α-amylase, β-amylase, glucoamylase, pullulanase, β-glucanase, hemicellulase, arabinosidase, mannanase, pectin hydrolase, pectate lyase, or a combination of these polysaccharases. In a related embodiment, the polysaccharase is a glucanase, preferably an expression product of a celZ or celY gene, and more preferably, derived from Erwinia chrysanthemi.

In one embodiment, the ethanologenic cells are exposed to a compound in an aqueous solution. For example, the fermentation media may be aqueous Luria broth (LB), variations thereof, other suitable medias, e.g., 1% CSL (see also those media described in the Examples) or media described in, e.g., Sambrook et al. or Ausubel et al., supra.

In another embodiment, the saccharide source from which ethanol is produced by the methods of the present invention is selected from the group consisting of cellooligosaccharide, lignocellulose, hemicellulose, cellulose, pectin, xylose, glucose, corn steep liquor, and any combination thereof.

In one aspect of the invention, the method of exposing fermenting ethanologenic cells to a nutrient compound is performed over a period of time. In a preferred embodiment, the period of time is between about 1 hour and about 96 hours. The exposure of fermenting ethanologenic bacteria to a compound may be an exposure at one time point only, at more than one time point, or continuously. In one embodiment, the exposure of a compound may be at several time points over a specific time period. For example, the compound may be added to the fermentation media, I) at the time of inoculation of the fermentation media by an ethanologenic cell, 2) at the time of inoculation followed by a second addition after either 8 or 12 hours of fermentation, or 3) at the time of inoculation followed by subsequent additions after 12 hours and 24 hours. The addition of the compound may be at any time point during the fermentation of the saccharide source.

In one embodiment, the nutrient compound is, e.g., acetaldehyde or pyruvic acid, and is added to a final concentration between about 0.1 and about 4.0 g/L. The nutrient compound is preferably of the formula R₃—C(═O)—R₁ where R₁ is H, OH or COOR₂, R₂ is H or C₁-C₅ alkyl, R₃ is C₁-C₅ alkyl, or C₁-C₅ alkenyl and therefore includes, acetaldehyde, pyruvate, succinate, citrate, isocitrate, glutamate, α-ketoglutarate, malate, a casamino acid, a yeast extract, or any combination thereof (including free powder forms and salts thereof). Concentrations intermediate to the ranges cited above are also intended to be within the scope of the present invention (ie., 0.15 g/L, 0.2 g/L, 0.25 g/L, 0.3 g/L, 0.35 g/L, 0.4 g/L, 0.45 g/L, 0.5 g/L, 0.55 g/L, 0.6 g/L, 0.65 g/L, 0.7 g/L, 0.8 g/L, 0.9 g/L, 1.0 g/L, 1.5 g/L, 2.0 g/L, 2.5 g/L, 3.0 g/L, 3.5 g/L, and 4.0 g/L). It will be appreciated that no more than routine experimentation is needed for determining or optimizing, using the methods disclosed herein, a concentration or concentration range for a given nutrient compound or combination of compounds. In a preferred embodiment, acetaldehyde may be added to a concentration of 0.1 g/L, 0.25 g/L, 0.5 g/L, or 0.75 g/L, or any combination thereof, during the fermentation process in order to achieve optimum production of ethanol and increased cell growth. To determine increased production of ethanol and increased growth of the cell by the addition of a compound, distilled water may be added instead a compound as a control.

In another embodiment, the method of increasing production of ethanol and growth of the ethanologenic cell by the addition of a nutrient compound to fermenting ethanologenic bacteria can be performed at a pH between 6 and 8. pH values intermediate to the ranges cited above are also intended to be within the scope of the present invention (e.g., 6.5, 7, and 7.5). In a preferred embodiment, the method of the instant invention is performed at a pH of about 6.5. The addition of a base, e.g., 2N KOH, to the fermentation medium can be used to maintain a specific pH during fermentation, as described in the examples.

Exposing a culture of fermenting cells to acetaldehyde can be performed at a temperature between about 20° C. and about 40° C. Temperatures intermediate to the ranges cited above are also intended to be within the scope of the present invention (e.g., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., and 39° C.). In a preferred embodiment, the method is performed at a temperature of about 35° C. Fermentation may be performed with agitation between about 50 and about 200 rpm of the fermentation medium after inoculation with the cells and exposure to a compound. Rates of agitation intermediate to the ranges cited above are also intended to be within the scope of the present invention (ie., 60, 70, 80, 90, 100, 110, 120, 130, 140, 150, 160,170, 180, and 190).

The present invention is also based, in part, on a fermentation reaction suitable for producing alcohol, e.g., ethanol. In one embodiment, the growth medium has a saccharide source, an ethanologenic cell, and an exogenous source of a compound, where the fermentation reaction is incubated under conditions sufficient for producing ethanol. For example, the saccharide source of the fermentation reaction can be selected from the group consisting of cellooligosaccharide, lignocellulose, hemicellulose, cellulose, pectin, xylose, glucose, and any combination thereof. An exogenous source of a compound may be any source obtained from outside of the fermentation reaction itself. The claimed fermentation reaction is also suitable for use in simultaneous saccharification and fermentation, where the degradation or depolymerization of a complex sugar and bioconversion of that sugar residue into ethanol by fermentation takes place contemporaneously in a single fermentation reaction of the present invention.

Furthermore, another aspect of this invention includes a growth medium suitable for use in an improved fermentation reaction. The growth medium contains a saccharide source as described above, a basal nutrient medium such as, for example, Luria broth or 1% CSL, minerals, and a nutrient compound. The growth medium of the invention is suitable for use in simultaneous saccharification and fermentation.

III. Potential Substrates for Bioconversion into Ethanol

One advantage of the invention is the ability to use a saccharide source that has been, heretofore, underutilized, and to increase the production of ethanol from that particular saccharide source. In addition, based on increased ethanol production, the amount of saccharide source used in a fermentation reaction can be reduced, thereby saving costs associated with the saccharide source while producing the same amount of ethanol.

A number of complex saccharide substrates may be used as a starting source for depolymerization and subsequent fermentation using the cells and methods of the invention. Ideally, a recyclable resource may be used in the SSF process. Mixed waste office paper is a preferred substrate (Brooks etal., (1995) Biotechnol. Progress. 11:619-625; Ingram et al., (1995) U.S. Pat. No. 5,424,202), and is much more readily digested than acid pretreated bagasse (Doran et al., (1994) Biotech. Bioeng. 44:240-247) or highly purified crystalline cellulose (Doran et al. (1993) Biotechnol. Progress. 9:533-538).

Glucanases, both endoglucanases and exoglucanases, contain a cellulose binding domain, and can be readily recycled for subsequent fermentations by harvesting the undigested cellulose residue using centrifugation (Brooks et al., (1995) Biotechnol. Progress. 11:619-625). By adding this residue with bound enzyme as a starter, ethanol yields (per unit substrate) can be increased to over 80% of the theoretical yield with a concurrent 60% reduction in fungal enzyme usage. Such approaches work well with purified cellulose, although the number of recycling steps may be limited with substrates with a higher lignin content. Other substrate sources that are within the scope of the invention include any type of processed or unprocessed plant material, e.g., lawn clippings, husks, cobs, stems, leaves, fibers, pulp, hemp, sawdust, newspapers, etc.

This invention is further illustrated by the following examples, which should not be construed as limiting.

### Exemplification

Materials and Methods

In general, the practice of the present invention employs, unless otherwise indicated, conventional techniques of chemistry, molecular biology, recombinant DNA technology, PCR technology, immunology, microbiology, or cell culture, which are within the skill of the art and are explained in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); PCR Handbook Current Protocols in Nucleic Acid Chemistry, Beaucage, Ed. John Wiley & Sons (1999); Antibodies:A Laboratory Manual, Harlow et al., C.S.H.L. Press, Pub. (1999); Bergey's Manual of Determinative Bacteriology, Kreig et al., Williams and Wilkins (1984), and Current Protocols in Molecular Biology, eds. Ausubel et al., Wiley Interscience (1998).

For additional techniques for using host cells in various industrial applications including a fermentation reaction for producing, e.g. ethanol, see, e.g., Barrios-Gonzalez et al., Biotechnol. Ann. Rev. 2:85-121 (1996); From Ethnomycology to Fungal Biotechnology: Exploiting from Natural Resources for Novel Products, Singh, J., Ed., Plenum Press, Pub. (1999); Manual of Industrial Microbiology and Biotechnology, Demain, A. Ed., Am. Soc. of Microbiology, Pub., (1999); Biomining: Theory, Microbes, and Industrial Processes, Rawlings, Ed., R. G. Landes Co., Pub. (1997); Biotechnology of Industrial Antibiotics, Vandamme, E., Ed., Marcel Dekker, Pub. (1984); Industrial Biotechnology, Malik, V., Ed., Science, Pub. (1992); Biotechnology and Food Ingredients, Goldberg et al., Ed., Aspen Publishers (1991); Biotechnology and Food Process Engineering, Schwartzberg et al., Ed., Marcel Dekker, Pub.(1990); and Food Biotechnology: Techniques and Applications, Mittal, G., Technomic Pub. Co. (1992).

Unless otherwise stated, the following materials and methods were used in the example that follows.

Fermentation Media

Two types of media were tested: 1% CSL and Luria broth (LB). Each contained 97 g of either xylose or glucose per liter. Luria Broth consists of, per liter: 10 g Difco® Tryptone, 5 g Difco® Yeast Extract and 5 g NaCl. The 1% CSL medium consists of Corn Steep Liquor (1% w/v) plus mineral salts (mineral salts per liter: 1 g of KH₂PO₄. 0.5 g of K₂HPO₄, 3.1 g of (NH₄)₂SO₄, 0.4 g of MgCl₂·6H₂), and 20 mg FeCl₃·6H₂O).

Media Preparation

Luria Broth was prepared by autoclaving a 2× nutrient stock containing, per liter: 20 g Difco® Tryptone, 10 g Difo® Yeast Extract, and 10 g NaCl. Sugars (xylose or glucose) were autoclaved separately. A 1% CSL medium was prepared as follows: Commercial Corn Steep Liquor (CSL; 50% dry weight/50% water) was diluted with tap water to make a 20X stock containing 100 g dry weight of Corn Steep Liquor/L and was adjusted to pH 7.2 with NaOH (50%). After sterilization by autoclaving, the 20X CSL stock was clarified by centrifugation at 5000×g for 10 minutes. Magnesium was added as a 100× stock solution (40 g/L MgCl₂·6H₂O). Iron was added as a 1000× stock solution made by dissolving 20 g FeCl₃·6H₂O in 175 mL of HCl and adjusting to 1 L with sterile water. Nitrogen, sulfur and phosphorus were added using a 20× stock solution containing the following (per liter): 20 g of KH₂PO₄, 10 g of K₂HPO₄ and 62 g of (NH₄)₂SO₄. Stock solutions containing minerals were sterilized separately by autoclaving. Final media was prepared by adding 750 mL of water to 1 L bottle containing 100 g of xylose or glucose. This solution was autoclaved, and stock solutions were added to provide a 1× concentration, and the final volume adjusted to 1 L. This broth was further diluted by the addition of supplements (acetaldehyde stock or distilled water) during fermentation with a resulting sugar content of 97 g/L.

Inoculum and Fermentation

Inocula were grown in same media used for fermentation and were prepared as follows. Three colonies from a fresh plate were used to inoculate seed cultures: 250-mL flask containing 100 mL medium. These cultures were grown with agitation (120 rpm) for 12-16 hours at 35° C. to a final OD₅₅₀ₙₘ of approximately 2.0-2.5. Cells were harvested by centrifugation at 5000×g for 5 minutes, and resuspended in fresh media to an OD₅₅₀ₙₘ of 0.1. The resulting suspension was distributed into 500-mL beakers containing 345 ml each of fermentation medium. Acetaldehyde was added as indicated. Batch fermentations (35° C., 100 rpm) were maintained at pH 6.5 by the automatic addition of 2N KOH. Cell mass, ethanol, and base addition (2N KOH) were recorded during 96 hours of incubation.

Addition of the Nutrient Compound Acetaldehyde

A fresh stock solution was prepared containing 35 g/L acetaldehyde in water. Three methods of acetaldehyde supplementation were investigated: 1) single additions of acetaldehyde at the time of inoculation; 2) addition of acetaldehyde at the time of inoculation followed by a second addition after 12 hours of fermentation for 1% CSL with xylose or after 8 h for 1% CSL with glucose and LB with xylose; and 3) addition of acetaldehyde (1% CSL with xylose medium only) at the time of inoculation followed by subsequent additions after 12 h and 24 h. Equivalent amounts of distilled water were added instead of the acetaldehyde in control experiments.

Other Nutrient Additives

Additives (e.g., as listed in Table 1) were dissolved in 5 mL di-H₂O. Solutions with pH lower than 5 were neutralized to pH 6.5 with 2N KOH. Nutritional supplements were then filter-sterilized (0.45 μm filter disk) directly into the fermentation vessel containing the culture. The final concentration of all additives in the culture medium was 2 g/L unless otherwise indicated.

Analytical Procedures

Cell mass was estimated by measuring OD₅₅₀ₙₘ using Baush & Lomb® Spectronic 70 spectrophotometer. Based on experimental determinations, 1 ml of cell suspension at 1.0 OD was found to contain 0.33 mg of cell dry weight. Ethanol was measured by gas chromatography using a Varian® 3400 CX gas chromatograph with 1-propanol as an internal standard.

### ExAMPLE 1

Methods for Improved Alcohol Production and Cell Growth in Escherichia

This example describes the effect of the addition of acetaldehyde to Luria broth (LB) and Xylose medium, 1% CSL and Xylose medium, and 1% CSL and glucose medium which have been inoculated with ethanologenic cells, i.e., recombinant Escherichia coli KO11. The effect of acetaldehyde on the production of ethanol and the growth of the ethanologenic cell, is described for each type of medium.

Tables 1, 3, and 5 show time to completion of fermentation, ethanol production, and volumetric productivity of the fermentation for all concentrations of the compound and the control. Maximum ethanol production is shown in grams/liter (g/L). The ethanol yield is shown in grams of ethanol/grams of added sugar. Maximum volumetric productivity is shown in grams ethanol/(liter)(hour). The average productivity is calculated form the start of fermentation to the completion of fermentation is grams ethanol/(liter)hour).

Tables 2, 4, and 6 show the initial growth of the cells (OD taken after the first 24 hours of fermentation) and the maximum cell density (the time which culture reaches the maximum OD value).

Culture Results Using 1% CSL and Xylose

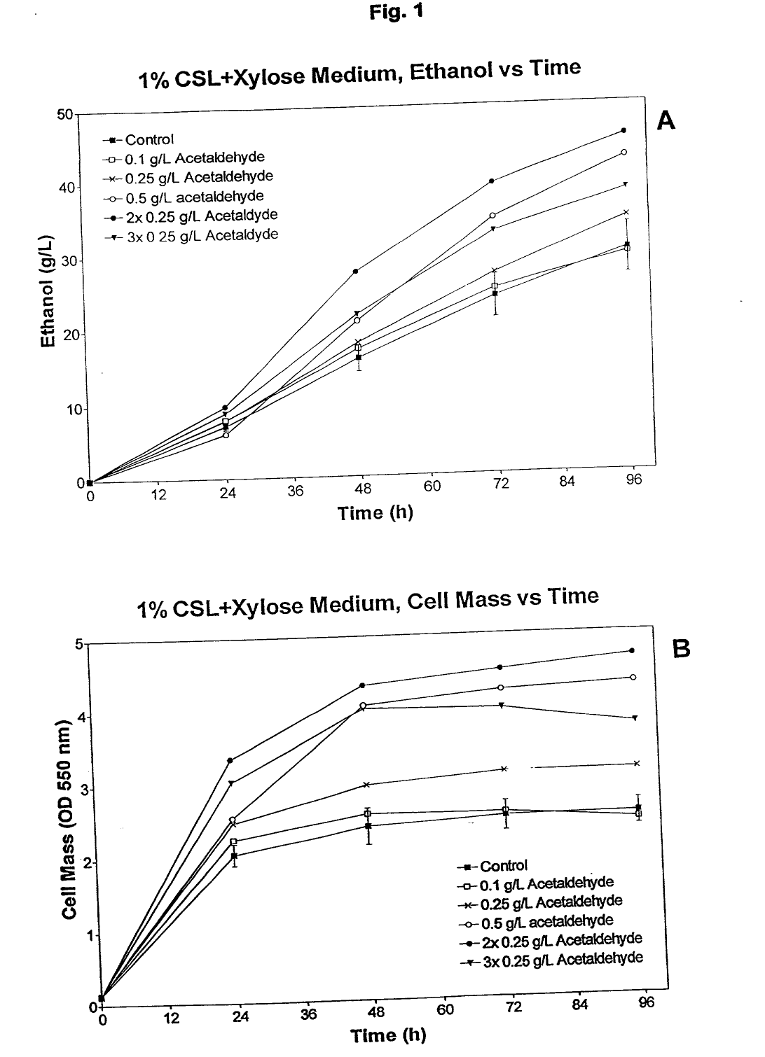

FIG. 1, panel A shows ethanol production in g/L over a period of 96 hours. FIG. 1, panel B shows cell mass (OD 550 nm), over 96 hours.

FIG. 1 and Table 1 show the results of the addition of acetaldehyde to 1% CSL and Xylose medium over a time period of 96 hours compared to a control (no addition of acetaldehyde). Acetaldehyde was added to the fermentation medium at five different concentrations and at five different time points. Where only one acetaldehyde value is provided, this amount of acetaldehyde was added at the start of fermentation only (i.e., 0.1 g/L acetaldehyde, 0.25 g/L acetaldehyde, and 0.5 g/L acetaldehyde). Where two additions are indicated, the first volume was added at the start of fermentation and the second was added after 12 hours of fermentation. For example, 2×0.25 g/L acetaldehyde refers to the addition of 0.25 g/L at the start of fermentation and 0.25 g/L after 12 hours. Where three additions are indicated, one was added at the start of fermentation, one after 12 hours and the third after 24 hours of fermentation. For example, 3×0.25 g/L refers to the addition of 0.25 g/L at the start, 0.25 g/L after 12 hours and 0.25 g/L after 24 hours.

TABLE 1
Culture Results Using 1% CSL Medium and Xylose (100 g/L)

| | | | | | | Volumetric | | | |
|---|---|---|---|---|---|---|---|---|---|
| | | | Ethanol Production | | | Productivity | | | |
| | | Time to | | % | | | % | | % |
| | Replicates | completion | Maximum | Control | Ethanol yield | Maximum | Control | Average | Control |
| Control | 11 | >96 h | 30.08 | 100.00 | 0.310 | 0.368 | 100.000 | 0.313 | 100.00 |
| 0.1 g/L Acetaldehyde | 2 | >96 h | 29.51 | 98.11 | 0.304 | 0.389 | 105.594 | 0.307 | 98.11 |
| 0.25 g/L Acetaldehyde | 2 | >96 h | 34.45 | 114.53 | 0.355 | 0.425 | 115.326 | 0.359 | 114.53 |
| 0.5 g/L Acetaldehyde | 2 | >96 h | 42.66 | 141.84 | 0.439 | 0.623 | 169.163 | 0.444 | 141.84 |
| 2 × 0.25 g/L Acetaldehyde | 2 | >96 h | 45.57 | 151.50 | 0.469 | 0.740 | 200.842 | 0.475 | 151.50 |
| 3 × 0.25 g/L Acetaldehyde | 2 | >96 h | 38.21 | 127.03 | 0.393 | 0.540 | 146.543 | 0.398 | 127.03 |
| Citrate | 2 | >96 h | 28.80 | 95.73 | 0.296 | 0.339 | 91.967 | 0.300 | 95.73 |
| Isocitrate | 2 | >96 h | 33.02 | 109.77 | 0.340 | 0.435 | 117.980 | 0.344 | 109.77 |
| Glutamate | 3 | 96 | 43.14 | 143.43 | 0.444 | 0.665 | 180.439 | 0.449 | 143.43 |
| Glutamate + 0.25 Acetaldehyde | 1 | 72 | 46.54 | 154.70 | 0.479 | 0.867 | 235.201 | 0.646 | 206.27 |
| Glutamate 0 0.5 Acetaldehyde | 1 | 72 | 44.97 | 149.51 | 0.463 | 0.818 | 221.931 | 0.625 | 199.34 |
| Oxaloacetate | 2 | >96 h | 23.61 | 78.49 | 0.243 | 0.270 | 73.268 | 0.246 | 78.49 |
| Aspartate | 2 | >96 h | 27.90 | 92.74 | 0.287 | 0.304 | 82.392 | 0.291 | 92.74 |
| Pyruvate + 0.25 Acetaldehyde | 2 | 72 | 45.71 | 151.94 | 0.470 | 0.920 | 249.715 | 0.635 | 202.59 |
| Pyruvate + 0.5 Acetaldehyde | 2 | 72 | 45.16 | 150.15 | 0.465 | 0.775 | 210.311 | 0.627 | 200.20 |
| Pyruvate | 5 | 72 | 44.42 | 147.66 | 0.457 | 0.792 | 215.013 | 0.617 | 196.88 |
| Alanine | 2 | >96 h | 21.66 | 72.02 | 0.223 | 0.240 | 65.176 | 0.226 | 72.02 |
| 4 g Pyruvate | 1 | 72 | 44.80 | 148.94 | 0.461 | 0.838 | 227.413 | 0.622 | 198.58 |
| 1 g Pyruvate | 1 | >96 h | 28.92 | 96.13 | 0.298 | 0.303 | 82.271 | 0.301 | 96.13 |
| 0.5 g Pyruvate | 1 | >96 h | 29.24 | 97.22 | 0.301 | 0.324 | 87.855 | 0.305 | 97.22 |
| 0.25 g Pyruvate | 1 | >96 h | 29.21 | 97.11 | 0.301 | 0.341 | 92.601 | 0.304 | 97.11 |
| A-KG + Succinate | 2 | 72 | 41.78 | 138.89 | 0.430 | 0.802 | 217.601 | 0.580 | 185.19 |
| a-KG | 2 | 72 | 42.32 | 140.70 | 0.436 | 0.866 | 234.952 | 0.588 | 187.59 |
| Succinate | 3 | >96 h | 35.00 | 116.35 | 0.360 | 0.400 | 108.536 | 0.365 | 116.35 |
| Fumarate + Malate | 1 | >96 h | 33.13 | 110.14 | 0.341 | 0.399 | 108.247 | 0.345 | 110.14 |
| Yeast Extract | 2 | 72 | 44.07 | 146.51 | 0.454 | 0.643 | 174.569 | 0.612 | 195.35 |
| Casamino Acids | 2 | 72 | 44.95 | 149.42 | 0.463 | 0.671 | 182.180 | 0.624 | 199.22 |
| Fumarate | 2 | >96 | 28.49 | 94.73 | 0.293 | 0.345 | 93.682 | 0.297 | 94.73 |
| Malate | 2 | >96 | 24.43 | 81.22 | 0.252 | 0.274 | 74.453 | 0.255 | 81.22 |

TABLE 2
Culture Results Using 1% CSL Medium and Xylose (100 g/L)

| | | Initial Growth | | | | |
|---|---|---|---|---|---|---|
| | | (OD @ 24 h) | | Maximum Cell Density | | |
| | Replicates | OD | % Control | Time (h) | OD | % Control |
| Control | 11 | 2.04 | 100.00 | 96 | 2.52 | 100.00 |
| 0.1 g/L Acetaldehyde | 2 | 2.25 | 110.04 | 48 | 2.56 | 101.92 |
| 0.25 g/L Acetaldehyde | 2 | 2.48 | 121.20 | 96 | 3.12 | 124.15 |
| 0.5 g/L Acetaldehyde | 2 | 2.55 | 124.64 | 96 | 4.31 | 171.13 |
| 2 × 0.25 g/L | 2 | 3.36 | 164.64 | 96 | 4.67 | 185.55 |
| Acetaldehyde | | | | | | |
| 3 × 0.25 g/L | 2 | 3.04 | 148.81 | 48 | 4.02 | 159.74 |
| Acetaldehyde | | | | | | |
| Citrate | 2 | 2.12 | 104.03 | 96 | 2.47 | 98.09 |
| Isocitrate | 2 | 2.17 | 106.01 | 96 | 3.01 | 119.43 |
| Glutamate | 3 | 3.69 | 180.49 | 96 | 5.04 | 200.16 |
| Glutamate + 0.25 | 1 | 4.03 | 197.30 | 96 | 6.13 | 243.62 |
| Acetaldehyde | | | | | | |
| Glutamate 0 0.5 | 1 | 4.10 | 200.52 | 96 | 5.98 | 237.83 |
| Acetaldehyde | | | | | | |
| Oxaloacetate | 2 | 1.65 | 80.80 | 96 | 2.26 | 89.89 |
| Aspartate | 2 | 1.76 | 86.27 | 72 | 2.45 | 97.20 |
| Pyruvate + 0.25 | 2 | 4.28 | 209.46 | 72 | 6.19 | 245.84 |
| Acetaldehyde | | | | | | |
| Pyruvate + 0.5 | 2 | 2.89 | 141.25 | 72 | 5.62 | 223.18 |
| Acetaldehyde | | | | | | |
| Pyruvate | 5 | 4.11 | 201.20 | 96 | 5.98 | 237.58 |
| Alanine | 2 | 1.66 | 81.34 | 48 | 1.73 | 68.82 |
| 4 g Pyruvate | 1 | 5.00 | 244.69 | 72 | 6.58 | 261.52 |
| 1 g Pyruvate | 1 | 2.12 | 103.56 | 72 | 2.69 | 106.93 |
| 0.5 g Pyruvate | 1 | 2.05 | 100.14 | 72 | 2.64 | 104.79 |
| 0.25 g Pyruvate | 1 | 1.98 | 97.04 | 96 | 2.38 | 94.41 |
| A-KG + Succinate | 2 | 3.52 | 172.27 | 72 | 5.52 | 219.40 |
| a-KG | 2 | 3.55 | 173.77 | 72 | 5.66 | 224.85 |
| Succinate | 3 | 2.11 | 103.40 | 96 | 3.12 | 124.13 |
| Fumarate + Malate | 1 | 1.83 | 89.38 | 96 | 2.68 | 106.70 |
| Yeast Extract | 2 | 3.75 | 183.56 | 96 | 4.83 | 192.03 |
| Casamino Acids | 2 | 4.31 | 210.82 | 96 | 5.34 | 212.03 |
| Fumarate | 2 | 1.86 | 91.16 | 96 | 2.60 | 103.15 |
| Malate | 2 | 1.71 | 83.93 | 96 | 2.42 | 96.31 |

As shown in Tables 1 and 2, the addition of 0.25 g/L acetaldehyde at the start of fermentation and after 12 hours of fermentation in 1% CSL and xylose resulted in the highest amount of ethanol production as compared to the control. In addition, this concentration of acetaldehyde also resulted in the greatest initial growth and maximum cell density as compared to a control.

Culture Results Using 1% CSL and Glucose

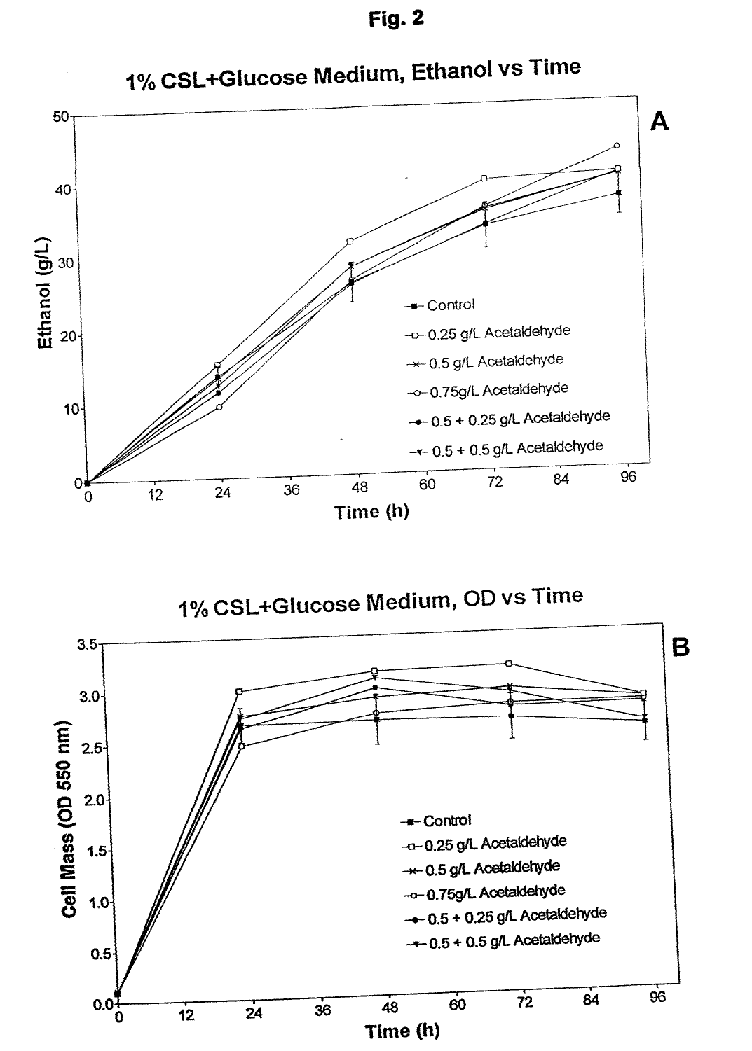

FIG. 2A shows ethanol production in g/L over a period of up to 96 hours. FIG. 2B shows cell mass (OD 550 nm), over 96 hours.

FIG. 2 and Table 3 show the results of the addition of acetaldehyde to 1% CSL and glucose medium over a time period of up to 96 hours compared to a control (no addition of acetaldehyde). Acetaldehyde was added to the fermentation medium at five different concentrations and time points. Where only one acetaldehyde value is provided, this amount of acetaldehyde was added at the start of fermentation only (i.e., 0.25 g/L acetaldehyde, 0.5 g/L acetaldehyde, and 0.75 g/L acetaldehyde). Where two additions are indicated, the first volume was added at the start of fermentation and the second was added after 8 hours of fermentation. For example, 0.5+0.25 g/L acetaldehyde refers to the addition of 0.5 g/L at the start of fermentation and 0.25 g/L after 8 hours, and 0.5+0.5/L acetaldehyde refers to the addition of 0.5 g/L at the start of fermentation and 0.5 g/L after 8 hours.

TABLE 3
Culture Results Using 1% CSL Medium and Glucose (100 g/L)

| | | | | | | Volumetric | | | |
|---|---|---|---|---|---|---|---|---|---|
| | | | Ethanol Production | | | Productivity | | | |
| | | Time to | | % | | | % | | % |
| | Replicates | completion | Maximum | Control | Ethanol yield | Maximum | Control | Average | Control |
| Control | 13 | 96 | 36.99 | 100.00 | 0.381 | 0.514 | 100.00 | 0.385 | 100.00 |
| 0.25 g/L Acetaldehyde | 1 | 72 | 40.31 | 108.97 | 0.415 | 0.678 | 132.01 | 0.560 | 145.29 |
| 0.5 g/L Acetaldehyde | 3 | 96 | 39.95 | 107.99 | 0.411 | 0.615 | 119.73 | 0.416 | 107.99 |
| 0.75 g/L Acetaldehyde | 1 | 96 | 43.44 | 117.43 | 0.447 | 0.699 | 136.11 | 0.452 | 117.43 |
| 0.5 + 0.25 g/L Acetaldehyde | 1 | 96 | 40.25 | 108.82 | 0.414 | 0.598 | 116.48 | 0.419 | 108.82 |
| 0.5 + 0.5 g/L Acetaldehyde | 1 | 96 | 40.03 | 108.22 | 0.412 | 0.659 | 128.25 | 0.417 | 108.22 |
| Aspartate | 1 | 72 | 41.79 | 112.96 | 0.430 | 0.759 | 147.72 | 0.580 | 150.61 |
| Oxaloacetate (max vp @ 24) | 1 | 72 | 39.72 | 107.37 | 0.409 | 0.712 | 138.48 | 0.552 | 143.16 |
| Citrate | 2 | 96 | 32.87 | 88.86 | 0.338 | 0.502 | 97.77 | 0.342 | 88.86 |
| Isocitrate | 2 | 72 | 33.95 | 91.77 | 0.349 | 0.588 | 114.38 | 0.471 | 122.36 |
| Glutamate (max vp @ 24) | 1 | 72 | 43.39 | 117.30 | 0.447 | 0.763 | 148.52 | 0.603 | 156.40 |
| a-ketoglutarate | 2 | 72 | 40.33 | 109.03 | 0.415 | 0.657 | 127.95 | 0.560 | 145.37 |
| Pyruvate (max vp @ 24) | 4 | 96 | 42.35 | 114.48 | 0.436 | 0.663 | 129.06 | 0.441 | 114.48 |
| Alanine (max vp @ 24) | 2 | 96 | 34.37 | 92.90 | 0.354 | 0.468 | 91.10 | 0.358 | 92.90 |
| 4 g Pyruvate (max vp @ 24) | 2 | 96 | 44.83 | 121.18 | 0.461 | 0.585 | 113.84 | 0.467 | 121.18 |
| Succinate (max vp @ 24) | 2 | 96 | 30.13 | 81.46 | 0.310 | 0.511 | 99.45 | 0.314 | 81.46 |
| Fumarate (max vp @ 24) | 2 | 96 | 32.92 | 88.98 | 0.339 | 0.559 | 108.88 | 0.343 | 88.98 |
| Malate (max vp @ 24) | 2 | 96 | 27.58 | 74.56 | 0.284 | 0.498 | 96.97 | 0.287 | 74.56 |

Stimulation with glucose was smaller than xylose, but present. Oxaloacetate stimulated more
with glucose, while 2-ketoglutarate stimulated more in xylose

TABLE 4
Culture Results Using 1% CSL Medium and Glucose (100 g/L)

| | | Initial Growth | | | | |
|---|---|---|---|---|---|---|
| | | (OD @ 24 h) | | Maximum Cell Density | | |
| | Replicates | OD | % Control | Time (h) | OD | % Control |
| Control | 13 | 2.67 | 100.00 | 48 | 2.69 | 100.00 |
| 0.25 g/L Acetaldehyde | 1 | 3.00 | 112.34 | 72 | 3.17 | 118.01 |
| 0.5 g/L Acetaldehyde | 3 | 2.77 | 103.55 | 72 | 2.96 | 110.00 |
| 0.75 g/L Acetaldehyde | 1 | 2.47 | 92.47 | 72 | 2.81 | 104.56 |
| 0.5 + 0.25 g/L Acetaldehyde | 1 | 2.64 | 98.90 | 48 | 2.99 | 111.40 |
| 0.5 + 0.5 g/L Acetaldehyde | 1 | 2.74 | 102.52 | 48 | 3.09 | 115.00 |
| Aspartate | 1 | 2.86 | 107.10 | 72 | 3.79 | 140.94 |
| Oxaloacetate (max vp @ 24) | 1 | 3.29 | 123.05 | 72 | 3.46 | 128.90 |
| Citrate | 2 | 2.43 | 90.97 | 72 | 2.58 | 95.99 |
| Isocitrate | 2 | 2.78 | 103.82 | 72 | 2.62 | 97.64 |
| Glutamate (max vp @ 24) | 1 | 2.86 | 107.10 | 72 | 3.79 | 140.94 |
| a-ketoglutarate | 2 | 3.29 | 123.05 | 72 | 3.46 | 128.90 |
| Pyruvate (max vp @ 24) | 4 | 3.46 | 129.39 | 72 | 3.83 | 142.47 |
| Alanine (max vp @ 24) | 2 | 2.08 | 77.97 | 96 | 2.68 | 99.80 |
| 4 g Pyruvate (max vp @ 24) | 2 | 3.84 | 143.44 | 72 | 4.07 | 151.63 |
| Succinate (max vp @ 24) | 2 | 2.69 | 100.47 | 72 | 2.66 | 98.99 |
| Fumarate (max vp @ 24) | 2 | 2.67 | 99.76 | 72 | 3.17 | 117.92 |
| Malate (max vp @ 24) | 2 | 3.19 | 119.49 | 72 | 3.39 | 126.18 |

As shown in Table 4, addition of 0.75 g/L of acetaldehyde at the start of fermentation in 1% CSL and glucose resulted in the highest amount of ethanol production as compared to the control. The addition of 0.25 g/L of acetaldehyde at the start of fermentation also resulted in the greatest initial growth and maximum cell density as compared to a control.

Culture Results Using LB and Xylose

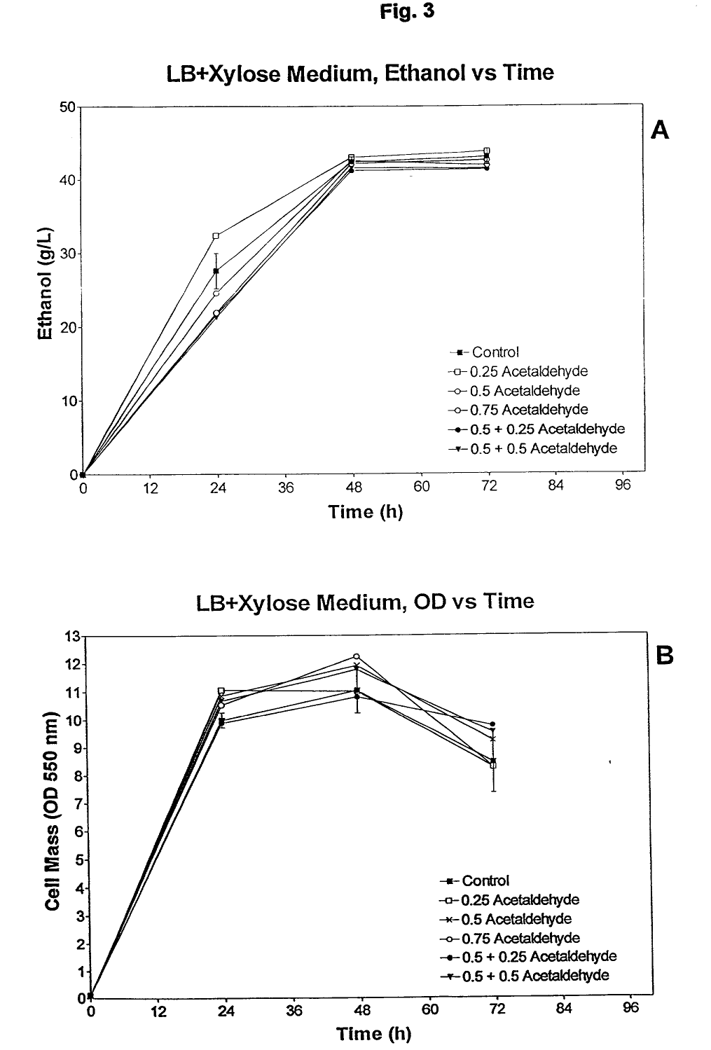

FIG. 3A shows ethanol production in g/L over 72 hours. FIG. 3B shows cell mass (OD 550 nm), over 72 hours.

FIG. 3 and Table 5 show the results of the addition of acetaldehyde to LB and xylose medium over a time period of 72 hours as compared to a control (no addition of acetaldehyde). Acetaldehyde was added to the fermentation medium at five different concentrations and time points. Where only one acetaldehyde value is provided, this amount of acetaldehyde was added at the start of fermentation only (ie., 0.25 g/L acetaldehyde, 0.5 g/L acetaldehyde, and 0.75 g/L acetaldehyde). Where two additions are indicated, the first volume was added at the start of fermentation and the second was added after 8 hours of fermentation. For example, 0.5+0.25 g/L acetaldehyde refers to the addition of 0.5 g/L at the start of fermentation and 0.25 g/L after 8 hours, and 0.5+0.5/L acetaldehyde refers to the addition of 0.5 g/L at the start of fermentation and 0.5 g/L after 8 hours.

TABLE 5
Culture Results Using Luria Broth Medium and Xylose (100 g/L)

| | | | | | | Volumetric | | | |
|---|---|---|---|---|---|---|---|---|---|
| | | | Ethanol Production | | | Productivity | | | |
| | | Time to | | % | | | % | | % |
| | Replicates | completion | Maximum | Control | Ethanol yield | Maximum | Control | Average | Control |
| Control | 5 | 48 | 43.08 | 100.00 | 0.433 | 1.152 | 100.00 | 0.897 | 100.00 |
| 0.25 g/L Acetaldehyde | 1 | 48 | 43.80 | 101.68 | 0.451 | 1.352 | 117.36 | 0.913 | 101.68 |
| 0.5 g/L Acetaldehyde | 2 | 48 | 42.59 | 98.88 | 0.438 | 1.027 | 89.20 | 0.887 | 98.88 |
| 0.75 g/L Acetaldehyde | 1 | 48 | 42.65 | 99.00 | 0.439 | 0.916 | 79.50 | 0.889 | 99.00 |
| 0.5 + 0.25 g/L Acetaldehyde | 2 | 48 | 41.40 | 96.11 | 0.426 | 0.904 | 78.52 | 0.863 | 96.11 |
| 0.5 + 0.5 g/L Acetaldehyde | 2 | 48 | 41.64 | 96.65 | 0.429 | 0.890 | 77.26 | 0.867 | 96.65 |
| a-ketoglutarate | 2 | 48 | 42.47 | 98.60 | 0.437 | 1.348 | 117.02 | 0.885 | 98.60 |
| Glutamate | 2 | 48 | 44.67 | 103.69 | 0.460 | 1.328 | 115.30 | 0.931 | 103.69 |
| 2 g Pyruvate | 2 | 48 | 44.35 | 102.96 | 0.457 | 1.284 | 111.47 | 0.924 | 102.96 |
| 4 g Pyruvate | 2 | 48 | 45.13 | 104.76 | 0.465 | 1.166 | 101.27 | 0.940 | 104.76 |

TABLE 6
Culture Results Using Luria Broth Medium and Xylose (100 g/L)

| | | Initial Growth | | | | |
|---|---|---|---|---|---|---|
| | | (OD at 24 h) | | Maximum Cell Density | | |
| | Replicates | OD | % Control | Time (h) | OD | % Control |
| Control | 5 | 9.99 | 100.00 | 48 | 11.04 | 100.00 |
| 0.25 g/L Acetaldehyde | 1 | 11.07 | 110.79 | 24 | 11.07 | 100.20 |
| 0.5 g/L Acetaldehyde | 2 | 10.86 | 108.77 | 48 | 11.92 | 107.97 |
| 0.75 g/L Acetaldehyde | 1 | 10.54 | 105.54 | 48 | 12.25 | 110.94 |
| 0.5 + 0.25 g/L Acetaldehyde | 2 | 9.89 | 99.02 | 48 | 10.81 | 97.92 |
| 0.5 + 0.5 g/L Acetaldehyde | 2 | 10.68 | 106.98 | 48 | 11.78 | 106.68 |
| a-ketoglutarate | 2 | 10.27 | 102.79 | 48 | 10.59 | 95.90 |
| Glutamate | 2 | 10.61 | 106.21 | 24 | 10.61 | 96.07 |
| 2 g Pyruvate | 2 | 11.30 | 113.09 | 24 | 11.30 | 102.29 |
| 4 g Pyruvate | 2 | 11.45 | 114.64 | 48 | 11.65 | 105.48 |

As shown in Table 3, addition of 0.5 g/L of acetaldehyde at the start of fermentation in LB+xylose resulted in the highest amount of ethanol production as compared to the control. The addition of 0.5 g/L of acetaldehyde at the start of fermentation results in the greatest initial growth and maximum cell density as compared to the control.

### EXAMPLE 2

Methods for Improved Alcohol Production and Cell Growth in Klebsiella

This example describes the effect of the addition of acetaldehyde to 1% CSL and xylose medium, which has been inoculated with ethanologenic, cells, i.e., Klebsiella oxytoca P2. The effect of acetaldehyde on the production of ethanol and the growth of the ethanologenic cell is presented in Table 7.

More particularly, Table 7 shows time to completion of fermentation, ethanol production and volumetric productivity of the fermentation for all concentrations of the compound and the control. Maximum ethanol production is shown in grams/liter (g/L). The ethanol yield is shown in grams of ethanol/grams of added sugar. Maximum volumetric productivity is shown in grams of ethanol/(liter)(hour). The average productivity is calculated form the start of fermentation to the completion of fermentation in grams ethanol/(liter)(hour). Table 7 also shows the initial growth of the cells (OD taken after the first 24 hours of fermentation) and the maximum cell density (the time which the culture reaches the maximum OD value at 48 hours).

Importantly, Table 7 shows the results of the addition of acetaldehyde to 1% CSL and xylose medium over a time period of 72 to 96 hours compared to a control (no addition of acetaldehyde) using an inoculum of the ethanologenic host Klebsiella oxytoca P2. Acetaldehyde was added to the fermentation medium at five different concentrations and time points. Where only one acetaldehyde value is provided, this amount of acetaldehyde was added at the start of fermentation only (i.e., 0.25 g/L acetaldehyde or 0.5 g/L). Where two or three additions are indicated, the first volume was added at the start of fermentation and additional doses of the nutrient compound were added after 8 hours of fermentation or at 8-hour intervals.

As shown in Table 7, addition of 0.5 g/L of acetaldehyde at the start of fermentation in CSL and xylose resulted in the highest amount of ethanol production as compared to the control. The addition of 0.5 g/L of acetaldehyde at the start of fermentation also resulted in increased initial growth and maximum cell density as compared to the control.

TABLE 7
Culture Results Using 1% CSL Medium and Xylose (100 g/L)

| | | Ethanol Production | | | Volumetric Productivity | | | |
|---|---|---|---|---|---|---|---|---|
| | Time to | | | | | % Control | | % Control |
| | completion | Maximum | % Control | Ethanol yield | Maximum | (based on Max) | Average | (based on Avg) |
| Control | >96 h | 23.69 | 100.00 | 0.244 | 0.34 | 100.0 | 0.247 | 100.00 |
| 0.25 g/L Acetaldehyde | >96 h | 25.43 | 107.35 | 0.262 | 0.47 | 135.46 | 0.265 | 107.35 |
| 0.5 g/L Acetaldehyde | >96 h | 26.53 | 111.98 | 0.273 | 0.51 | 147.11 | 0.276 | 111.98 |
| 2 × 0.25 g/L | >96 h | 25.56 | 107.88 | 0.263 | 0.44 | 126.68 | 0.266 | 107.88 |
| Acetaldehyde | | | | | | | | |
| 3 × 0.25 g/L | >96 h | 26.14 | 110.32 | 0.269 | 0.47 | 135.55 | 0.272 | 110.32 |
| Acetaldehyde | | | | | | | | |

| | Initial Growth (OD @ 24 h) | | Maximum Cell Density | | |
|---|---|---|---|---|---|
| | OD | % Control | Time (h) | OD | % Control |
| Control | 3.77 | 100.00 | 72.00 | 5.07 | 100.00 |
| 0.25 g/L Acetaldehyde | 3.54 | 93.90 | 72.00 | 5.68 | 111.96 |
| 0.5 g/L Acetaldehyde | 2.61 | 69.39 | 96.00 | 5.89 | 116.26 |
| 2 × 0.25 g/L Acetaldehyde | 3.34 | 88.59 | 96.00 | 5.30 | 104.50 |
| 3 × 0.25 g/L Acetaldehyde | 3.60 | 95.66 | 72.00 | 5.40 | 106.51 |

Equivalents

Those skilled in the art will recognize, or be able to ascertain using no more than routine experimentation, many equivalents to the specific embodiments of the invention described herein. Such equivalents are intended to be encompassed by the following claims. Moreover, any number of genetic constructs, host cells, and methods described in U.S. Pat. Nos. 5,821,093; 5,482,846; 5,424,202; 5,028,539; 5,000,000; 5,487,989, 5,554,520, and 5,162,516, may be employed in carrying out the present invention and are hereby incorporated by reference.

What is claimed:

1. A method for increasing production of alcohol from a saccharide source by an alcohologenic cell comprising,
contacting a saccharide source with an alcohologenic cell, and
exposing said cell to at least one compound of formula I 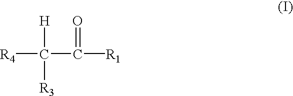
wherein;
R₁ is H, OH or COOR₂;
R₂ is H or alkyl;
R₃ is H, NH₂, alkyl or alkenyl;
R₄is H, alkyl, alkenyl, or a side chain of a naturally occurring amino acid; and
salts thereof,
wherein said exposing results in the increased production of alcohol by the alcohologenic cell as compared to a control.

2. A method for increasing growth of a cell comprising,
contacting a cell with a saccharide source, and
exposing said cell to at least one compound of formula I, 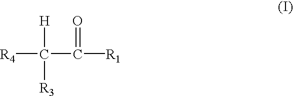
wherein;
R₁ is H, OH or COOR₂;
R₂ is H or alkyl;
R₃ is H, NH₂, alkyl or alkenyl;
R₄ is H, alkyl, alkenyl, or a side chain of a naturally occurring amino acid; and
salts thereof;
wherein said exposing results in the increased growth of said cell as compared to a control.

3. The method of claim 1 or 2, wherein said compound of formula I is selected from the group consisting of lower aliphatic aldehydes, lower aliphatic α-keto carboxylic acids, lower aliphatic dicarboxylic acids, amino acids, and salts of any of said acids.

4. The method of claim 1, wherein said alcohol is ethanol and said alcohologenic cell is an ethanologenic cell.

5. The method of claim 2, wherein said cell is an ethanologenic cell.

6. The method of claim 4 or claim 5, wherein said cell is selected from the family Enterobacteriaceae.

7. The method of claim 6, wherein said cell is Escherichia or Klebsiella.

8. The method of claim 7, wherein said cell is a recombinant cell.

9. The method of claim 8, wherein said cell is selected from the group consisting of E. coli KO4 (ATCC 55123), E. coli KO11 (ATCC 55124), E. coli KO12 (ATCC 55125), K. oxytoca M5A1, K. oxytoca P2 (ATCC 55307), and LY01 (ATCC______).

10. The method of claim 4, wherein said compound of formula I is selected from the group consisting of acetaldehyde, pyruvate, succinate, isocitrate, glutamate, α-ketoglutarate, casarnino acids, and yeast extract.

11. The method of claim 10, wherein said compound of formula I is acetaldehyde.

12. The method of claim 10, wherein said compound of formula I is pyruvate.

13. The method of claim 10, wherein said compound of formula I is glutamate.

14. The method of claim 5, wherein said compound of formula I is selected from the group consisting of acetaldehyde, pyruvate, succinate, citrate, isocitrate, glutamate, α-ketoglutarate, malate, casamino acids, and yeast extract.

15. The method of claim 14, wherein said compound of formula I is acetaldehyde.

16. The method of claim 14, wherein said compound of formula I is pyruvate.

17. The method of claim 14, wherein said compound of formula I is glutamate.

18. The method of claim 10 or 14, wherein said cell is exposed to glutamate and acetaldehyde.

19. The method of claim 10 or 14, wherein said cell is exposed to pyruvate and acetaldehyde.

20. The method of claim 10 or 14, wherein said cell is exposed to fumarate and malate.

21. The method of claim 10 or 14, wherein said cell is exposed to α-ketoglutarate and succinate.

22. The method of claim 1 or 2, further comprising providing said cell in an aqueous solution.

23. The method of claim 1 or 2, wherein said saccharide source is selected from the group consisting of cellooligosaccharide, lignocellulose, hemicellulose, cellulose, pectin, xylose, glucose, and any combination thereof.

24. The method of claim 1 or 2, wherein said cell is exposed to said compound of formula I for a period of time between about 1 and about 96 hours.

25. The method of claim 1 or 2, wherein said method is performed at a pH between about 6 and about 8.

26. The method of claim 25, wherein said method is performed at a pH of about 6.5.

27. The method of claim 1 or 2, wherein said method is performed at a temperature between about 20° and about 40° C.

28. The method of claim 27, wherein said method is performed at a temperature of about 35° C.

29. The method of claim 1 or 2, wherein said compound is present at a concentration between about 0.1 and about 4.0 g/L.

30. The method of claim 1 or 2, further comprising exposing said cell to said compound more than once.

31. The method of claim 1 or 2, further comprising exposing said cell to two or more different compounds of formula I.

32. The method of claim 31, wherein said exposing of said cell to said compound is performed at time intervals between about 1 hour and about 24 hours.

33. The method of claim 1 or 2, further comprising agitating said cell, said saccharide source, and said compound between about 50 rpm and about 200 rpm.

34. The method of claim 2, wherein said increased growth is indicated by increased cell density or decreased cell replication time.

35. The method of claim 34, wherein said increased cell density is indicated by an optical density of between about 2 and about 3 at 550 nm after 24 hours.

36. The method of claim 4, wherein said increased production of ethanol is indicated by an increase in volumetric productivity.

37. The method of claim 36, wherein said volumetric productivity is between about 0.3 g/L and about 0.5 g/L

38. The method of claim 1 or 2, wherein said method is performed in a fermentor vessel.

39. The method of claim 38, wherein said cell and said saccharide source are provided in an aqueous solution.

40. The method of claim 39, wherein said aqueous solution comprises a fermentation medium.

41. The method of claim 40, wherein said fermentation medium comprises Luria broth or CSL broth.

42. The method of claim 1 or 2, wherein said method is suitable for simultaneous saccharification and fermentation.

43. A growth medium suitable for use in an improved fermentation process comprising:
a saccharide source;
a basal nutrient medium, and
at least one compound of formula I, 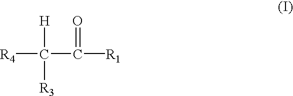
wherein;
R₁ is H, OH or COOR₂;
R₂ is H or alkyl;
R₃ is H, NH₂, alkyl or alkenyl;
R₄ is H, alkyl, alkenyl, or a side chain of a naturally occurring amino acid; and
salts thereof.

44. The growth medium of claim 43, wherein said saccharide source is selected from the group consisting of cellooligosaccharide, lignocellulose, hemicellulose, cellulose, pectin, xylose, glucose, corn steep liquor, and any combination thereof.

45. The growth medium of claim 43, wherein said basal nutrient medium is Luria broth or CSL broth.

46. The growth medium of claim 43, wherein said medium is suitable for use in simultaneous saccharification and fermentation.

47. The growth medium of claim 43, wherein said growth medium is packaged with instructions for use.

48. A fermentation reaction mixture suitable for producing ethanol comprising,
a growth medium having a saccharide source,
an ethanologenic cell, and
an exogenous source of at least one compound of formula I, 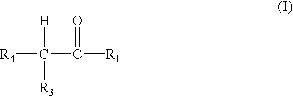
wherein;
R₁ is H, OH or COOR₂;
R₂ is H or alkyl;
R₃ is H, NH₂, alkyl or alkenyl;
R₄ is H, alkyl, alkenyl, or a side chain of a naturally occurring ammo acid; and
salts thereof.

49. The fermentation reaction mixture of claim 48, wherein said saccharide source is selected from the group consisting of cellooligosaccharide, lignocellulose, hemicellulose, cellulose, pectin, xylose, glucose, corn steep liquor, and any combination thereof.

50. The fermentation reaction mixture of claim 48, wherein said ethanologenic cell is from the family Enterobacteriaceae.

51. The fermentation reaction mixture of claim 48, wherein said reaction mixture is suitable for use in simultaneous saccharification and fermentation.

52. The growth medium of claim 43, wherein said compound of formula I is selected from the group consisting of acetaldehyde, pyruvate, succinate, citrate, isocitrate, glutamate, α-ketoglutarate, malate, fumarate, a yeast extract, and a casamino acid.

53. The fermentation reaction mixture of claim 48, wherein said compound of formula I is selected from the group consisting of acetaldehyde, pyruvate, succinate, isocitrate, glutamate, α-ketoglutarate, and a casamino acid.

54. The growth medium of claim 52, wherein said compound of formula I is selected from the group consisting of acetaldehyde, pyruvate, succinate, citrate, isocitrate, glutamate, α-ketoglutarate, and malate.

55. The fermentation reaction mixture of claim 53, wherein said compound of formula I is selected from the group consisting of acetaldehyde, pyruvate, succinate, isocitrate, glutamate, and α-ketoglutarate.
